(19) United States
(12) Patent Application Publication
(10) Pub. No.: US 2011/0206722 A1
(43) Pub. Date: Aug. 25, 2011

(54) Pharmaceutical Composition For Purifying Blood Vessels And Preparation Method Thereof

(76) Inventors: Zhizheng Du, Beijing (CN); Jiankui Tang, Beijing (CN)

(21) Appl. No.: 13/058,971

(22) PCT Filed: Nov. 28, 2008
(86) PCT No.: PCT/CN2008/073237

§ 371 Date: Feb. 14, 2011

(30) Foreign Application Priority Data
Nov. 14, 2008 (CN) 200810172194.8

(51) Int. Cl. A61K 36/06; A61K 36/258; A61P 9/00

(52) U.S. Cl. 424/195.15; 424/728

(57) ABSTRACT

A pharmaceutical composition includes Folium Isatidis 10-30 parts, Semen Cassiae 15-35 parts, Flos Inulae 10-20 parts, Flos Carthami 15-30 parts, Flos Chrysanthemi 10-30 parts, Herba Apocyni Veneti 20-50 parts, Ginkgo flavone 20-40 parts, emodin 20-35 parts, crataegin 30-50 parts, gypenoside 15-55 parts, Panax Notoginsenoside 20-60 parts. This composition can improve the level of high density lipoprotein in blood, clean up blockage of vein, soften vein, and reduce viscosity of blood.

### TECHNICAL FIELD

The present invention relates to a plant extract composition and a preparation method thereof. More particularly, the present invention relates to a plant extract composition capable of purifying blood vessels and a preparation method thereof.

### BACKGROUND ART

With the development in the economic society, there are many changes in the constitutions of people's diets, where more meat, milk, and egg products are consumed, resulting in more fat, protein, cholesterol, and the like being absorbed. However, when disturbance of lipid metabolism in the body occurs due to various reasons, “rubbish” in blood vessels, such as excessive cholesterol and triglycerides, will accumulate in the body, thus causing atherosclerosis and blockage of arteries.

In 1985, the American scholars M. S. Brown and J. L. Goldstein found that there is a high-density lipoprotein (HDL) in human body's blood, which can facilitates the breakdown of excessive cholesterol and triglycerides in the blood by the liver, reverses endothelial dysfunction in blood vessels, suppresses endothelial cell apoptosis, and prevents oxidation of a low-density lipoprotein (LDL) and aggregation of platelet. However, it remains very difficult to increase HDL in the body to a level capable of treating hyperlipidemia at present. A Romanian professor has extracted a kind of substance known as H3 from the drug procaine, which can increase HDL by about 34%. However, due to its severe toxicity, the wide use of this substance is limited.

When the blockage of blood vessels is still not serious and before no symptoms appear, it is very difficult to find that accumulation of the “rubbish” in blood vessels has started. Only when symptoms of cardiovascular and cerebrovascular diseases appear, the severity thereof can be realized; at this time, however, it has become less easy to clean up the blockage in the blood vessels. Usually, one method is to perform a surgery, such as bypass or stenting procedures, while the pain and risk of the surgery are well known; and another method is to use a drug, where for hyperlipidemia diseases, a type of drugs called statins are more frequently used at present. This type of drugs has good therapeutic effects, but also has some toxicity to the liver and muscles. Moreover, the use of drugs is undesirable and often results in poor compliance.

### SUMMARY OF THE INVENTION

An object of the present invention is to provide a novel plant extract composition functioning as a scavenger of blood vessels and a preparation method thereof.

On one embodiment of this invention, a plant extract composition functioning as a scavenger of blood vessels is mainly composed of plant raw materials and an extract, wherein the plant raw materials comprise the following components according to parts by weight: 10-30 parts of Folium Isatidis, 15-35 parts of Semen Cassiae, 10-20 parts of Flos Inulae, 15-30 parts of Flos Carthami, 10-30 parts of Flos Dendranthemae, and 20-50 parts of Folium Apocyni Veneti; and the extract comprises the following components according to parts by weight: 20-40 parts of Ginkgo flavone, 20-35 parts of emodin, 30-50 parts of crataegin, 15-55 parts of gypenoside, and 20-60 parts of Panax notoginseng saponins.

In a preferred embodiment of this invention, a plant extract composition is provided, wherein the plant raw materials comprise the following components according to parts by weight: 15-25 parts of Folium Isatidis, 20-30 parts of Semen Cassiae, 12-18 parts of Flos Inulae, 20-30 parts of Flos Carthami, 15-25 parts of Flos Dendranthemae, and 30-40 parts of Folium Apocyni Veneti; and the extract comprises the following components according to parts by weight: 25-35 parts of Ginkgo flavone, 25-35 parts of emodin, 30-40 parts of crataegin, 20-40 parts of gypenoside, and 30-50 parts of Panax notoginseng saponins.

In another preferred embodiment of this invention, in addition to the above components, the plant extract composition further comprises the following raw materials according to parts by weight: 10-30 parts of lecithin, 10-30 parts of black Auricularia, and 20-80 parts of solid acetic acid, wherein the lecithin is an extract, the solid acetic acid is extracted from plants and thus it also belongs to an extract in this invention, and black Auricularia is also classified as a plant raw material in this invention although it belongs to a fungi. The addition of lecithin, black Auricularia, and solid acetic acid may further soften blood vessels, prevent vascular sclerosis, and accelerate vascular reparation.

A dry final product, prepared from said plant extract composition by the preparation method below, can be prepared into capsule, tablet, powder, or granule, with or without the addition of suitable adjuvants or carriers.

Another object of the present invention is to provide a method for preparing the plant extract composition.

A method for preparing the plant extracts composition, comprising the following steps:
a) pulverizing: weighing the plant raw materials according to parts by weight respectively and pulverizing them;
b) soaking: the pulverized plant raw materials from step a) are placed into a sealed container, and then water is added thereto in a solid/liquid ratio of 1:4 by weight, and the resultant is soaked for 2-5 h;
c) ultrasonic extracting: the plant raw materials and soak solution obtained from step b) are extracted in an ultrasonic tank, where the temperature was raised from a room temperature to no greater than 40° C., and the extraction time was 50-60 min;
d) filtrating and separating: the mixed solution obtained from step c) is filtered to obtain a filtrate and a residue, the residue is subjected to centrifugal filtration to obtain a secondary filtrate, and both the filtrates are combined into a final filtrate;
e) adjusting pH: the final filtrate is adjusted to having a pH of 7.5-7.7;
f) concentrating: the final filtrate is concentrated to a concentrated product having a content of 75-80%;
g) adding a plant extract: the plant extract is weighted according to parts by weight, added into the concentrated product and mixed homogeneously; and
h) drying: the mixture obtained from step g) is dried to provide a dry final product.

In step c), the stirring rate in the ultrasonic tank is 1500 rpm, the ultrasonic power is 1500 W, and the ultrasonic interval is 1.5 min.

In said step h), vacuum drying or microwave drying may be used. In the case of vacuum drying, the influence of air on the product is reduced, the drying temperature is low, the drying rate is fast, and the contacting chance between the product and air is decreased, as well as contamination and oxidative deterioration are avoided, and the product is easily pulverized.

In the case of microwave drying, the material to be dried is radiated by a microwave generator, and when the microwave is irradiated to the inside of the material, polar molecules such as water move synchronously with the microwave frequency, so that frictional heat is generated instantaneously in the material, and thus the temperatures on the surface and inside of the material are increased simultaneously, then a large amount of the water molecules escape outside, hereby the drying of the material is achieved. The microwave drying has a high drying rate, short drying time, and uniform heating, thus it is very suitable for drying and sterilizing the raw materials of Chinese traditional medicines. The volatile and aromatic substances in the raw materials are less lost, because the drying time is 10- or 100-fold of that of conventional heating.

The dry final product made from the plant extract composition according to the present invention may be pulverized and prepared into a desired formulation such as capsule, tablet, powder, or granule, optionally with the addition of suitable adjuvants or carriers. Each of these formulations may be prepared according to conventional methods in the pharmaceutical field.

The plant extract composition of this invention is prepared by employing ultrasonic to extract the plant raw materials, and during preparing, no chemical reagent is used, thus natural active ingredients of the plants are remained so that various natural flavones, soaps, phospholipids and various enzymes are contained therein. After entering the blood vessels, the plant extract composition of the invention can increase the level of HDL in blood, and efficiently and quickly decompose and dissolve the lipids and blood deposits in blood. Simultaneously, it can dilate and soften the blood vessels, restore the tension and elasticity of the blood vessels, and repair the blood vessels to reduce the vascular resistance; and can also decrease the blood viscosity, purify the blood, and eliminate hazardous substances, rubbish and toxins in the blood vessels from the body, actually functioning as a scavenger of blood vessels.

### DETAILED DESCRIPTION OF THE INVENTION

### Embodiment 1

A plant extract composition functioning as a scavenger of blood vessels was prepared according to the following steps:
a) pulverizing: the plant raw materials were weighted according to parts by weight, respectively: 10 parts of Folium Isatidis, 35 parts of Semen Cassiae, 10 parts of Flos Inulae, 15 parts of Flos Carthami, 10 parts of Flos Dendranthemae, and 50 parts of Folium Apocyni Veneti, and pulverized;
b) soaking: the pulverized plant raw materials in step a) were placed into a sealed container, and then water was added thereto in a solid/liquid ratio of 1:4 by weight, and the resultant was soaked for 3 hours;
c) ultrasonic extracting: the plant raw materials and soak solution obtained from step b) were extracted in an ultrasonic tank, where the temperature was raised from a room temperature to 30° C., and the extraction time was 60 minutes;
d) filtrating and separating: the mixed solution obtained from step c) was filtered to obtain a filtrate and a residue, the residue was subjected to centrifugal filtration to obtain a secondary filtrate, and both the filtrates were combined into a final filtrate;
e) adjusting pH: the final filtrate was adjusted to having a pH of 7.5;
f) concentrating: the final filtrate was concentrated to a concentrated product having a content of 75%;
g) adding a plant extract: the plant extract was weighted according to parts by weight: 40 parts of Ginkgo flavone, 20 parts of emodin, 30 parts of crataegin, 15 parts of gypenoside, and 60 parts of Panax notoginseng saponins, and added into the concentrated product and mixed homogeneously; and
h) drying: the mixture obtained from step g) was dried under vacuum to provide a dry final product.

### Embodiment 2

A plant extract composition functioning as a scavenger of blood vessels was prepared according to the following steps:
a) pulverizing: the plant raw materials were weighted according to parts by weight, respectively: 30 parts of Folium Isatidis, 15 parts of Semen Cassiae, 20 parts of Flos Inulae, 30 parts of Flos Carthami, 30 parts of Flos Dendranthemae, and 20 parts of Folium Apocyni Veneti, and pulverized;
b) soaking: the pulverized plant raw materials in step a) were placed into a sealed container, and then water was added thereto in a solid/liquid ratio of 1:4 by weight, and the resultant was soaked for 2 hours;
c) ultrasonic extracting: the plant raw materials and soak solution obtained from step b) were extracted in an ultrasonic tank, where the temperature was raised from a room temperature to 35° C., and the extraction time was 50 minutes;
d) filtrating and separating: the mixed solution obtained from step c) was filtered to obtain a filtrate and a residue, the residue was subjected to centrifugal filtration to obtain a secondary filtrate, and both the filtrates were combined into a final filtrate;
e) adjusting pH: the final filtrate was adjusted to having a pH of 7.6;
f) concentrating: the final filtrate was concentrated to a concentrated product having a content of 80%;
g) adding a plant extract: the plant extract was weighted according to parts by weight: 20 parts of Ginkgo flavone, 35 parts of emodin, 50 parts of crataegin, 55 parts of gypenoside, and 20 parts of Panax notoginseng saponins, and added into the concentrated product and mixed homogeneously; and
h) drying: the mixture obtained from step g) was dried under vacuum to provide a dry final product.

### Embodiment 3

A plant extract composition functioning as a scavenger of blood vessels was prepared according to the following steps:
a) pulverizing: the plant raw materials were weighted according to parts by weight, respectively: 15 parts of Folium Isatidis, 30 parts of Semen Cassiae, 12 parts of Flos Inulae, 30 parts of Flos Carthami, 25 parts of Flos Dendranthemae, and 40 parts of Folium Apocyni Veneti, and pulverized, additionally, 10 parts of black Auricularia was weighted and pulverized;
b) soaking: the pulverized plant raw materials and black Auricularia in step a) were placed into a sealed container, and then water was added thereto in a solid/liquid ratio of 1:4 by weight, and the resultant was soaked for 4 hours;
c) ultrasonic extracting: the plant raw materials and soak solution obtained from step b) were extracted in an ultrasonic tank, where the temperature was raised from a room temperature to 40° C., and the extraction time was 50 minutes;
d) filtrating and separating: the mixed solution obtained from step c) was filtered to obtain a filtrate and a residue, the residue was subjected to centrifugal filtration to obtain a secondary filtrate, and both the filtrates were combined into a final filtrate;
e) adjusting pH: the final filtrate was adjusted to having a pH of 7.7;
f) concentrating: the final filtrate was concentrated to a concentrated product having a content of 80%;
g) adding a plant extract: the plant extract was weighted according to parts by weight: 25 parts of Ginkgo flavone, 25 parts of emodin, 30 parts of crataegin, 20 parts of gypenoside, and 50 parts of Panax notoginseng saponins, as well as 30 parts of lecithin and 20 parts of solid acetic acid, and added into the concentrated product and mixed homogeneously; and
h) drying: the mixture obtained from step g) was dried under microwave to provide a dry final product.

### Embodiment 4

A plant extract composition functioning as a scavenger of blood vessels was prepared according to the following steps:
a) pulverizing: the plant raw materials were weighted according to parts by weight, respectively: 20 parts of Folium Isatidis, 25 parts of Semen Cassiae, 15 parts of Flos Inulae, 25 parts of Flos Carthami, 20 parts of Flos Dendranthemae, and 30 parts of Folium Apocyni Veneti, and pulverized, additionally, 20 parts of black Auricularia was weighted and pulverized;
b) soaking: the pulverized plant raw materials and black Auricularia in step a) were placed into a sealed container, and then water was added thereto in a solid/liquid ratio of 1:4 by weight, and the resultant was soaked for 3 hours;
c) ultrasonic extracting: the plant raw materials and soak solution obtained from step b) were extracted in an ultrasonic tank, where the temperature was raised from a room temperature to 30° C., and the extraction time was 60 minutes;
d) filtrating and separating: the mixed solution obtained from step c) was filtered to obtain a filtrate and a residue, the residue was subjected to centrifugal filtration to obtain a secondary filtrate, and both the filtrates were combined into a final filtrate;
e) adjusting pH: the final filtrate was adjusted to having a pH of 7.5;
f) concentrating: the final filtrate was concentrated to a concentrated product having a content of 80%;
g) adding a plant extract: the plant extract was weighted according to parts by weight: 30 parts of Ginkgo flavone, 30 parts of emodin, 35 parts of crataegin, 25 parts of gypenoside, and 40 parts of Panax notoginseng saponins, as well as 20 parts of lecithin and 80 parts of acetic acid solid, and added into the concentrated product and mixed homogeneously; and
h) drying: the mixture obtained from step g) was dried under microwave to provide a dry final product.

### Embodiment 5

A plant extract composition functioning as a scavenger of blood vessels was prepared according to the following steps:
a) pulverizing: the plant raw materials were weighted according to parts by weight, respectively: 25 parts of Folium Isatidis, 20 parts of Semen Cassiae, 18 parts of Flos Inulae, 20 parts of Flos Carthami, 15 parts of Flos Dendranthemae, and 30 parts of Folium Apocyni Veneti, and pulverized;
b) soaking: the pulverized plant raw materials in step a) were placed into a sealed container, and then water was added thereto in a solid/liquid ratio of 1:4 by weight, and the resultant was soaked for 5 hours;
c) ultrasonic extracting: the plant raw materials and soak solution obtained from step b) were extracted in an ultrasonic tank, where the temperature was raised from a room temperature to 40° C., and the extraction time was 50 minutes;
d) filtrating and separating: the mixed solution obtained from step c) was filtered to obtain a filtrate and a residue, the residue was subjected to centrifugal filtration to obtain a secondary filtrate, and both the filtrates were combined into a final filtrate;
e) adjusting pH: the final filtrate was adjusted to have a pH of 7.6;
f) concentrating: the final filtrate was concentrated to a concentrated product having a content of 80%;
g) adding a plant extract: the plant extract was weighted according to parts by weight: 35 parts of Ginkgo flavone, 35 parts of emodin, 40 parts of crataegin, 40 parts of gypenoside, and 30 parts of Panax notoginseng saponins, and added into the concentrated product and mixed homogeneously; and
h) drying: the mixture obtained from step g) was dried under vacuum to provide a dry final product.

### Embodiment 6

A plant extract composition functioning as a scavenger of blood vessels was prepared according to the following steps:
a) pulverizing: the plant raw materials were weighted according to parts by weight, respectively: 20 parts of Folium Isatidis, 25 parts of Semen Cassiae, 16 parts of Flos Inulae, 25 parts of Flos Carthami, 20 parts of Flos Dendranthemae, and 35 parts of Folium Apocyni Veneti, and pulverized, and then 30 parts of black Auricularia was weighted and pulverized;
b) soaking: the pulverized plant raw materials in step a) were placed into a sealed container, and then water was added thereto in a solid/liquid ratio of 1:4 by weight, and they were soaked for 3 hours;
c) ultrasonic extracting: the plant raw materials and soak solution obtained in step b) were extracted in a ultrasonic tank, where the temperature was raised from a room temperature to 40° C., and the extraction time was 50 minutes;
d) filtrating and separating: the mixed solution obtained from step c) was filtered to obtain a filtrate and a residue, the residue was subjected to centrifugal filtration to obtain a secondary filtrate, and both the filtrates were combined into a final filtrate;
e) adjusting pH: the final filtrate was adjusted to have a pH of 7.7;
f) concentrating: the final filtrate was concentrated to a concentrated product having a content of 80%;
g) adding a plant extract: the plant extract was weighted according to parts by weight: 30 parts of Ginkgo flavone, 30 parts of emodin, 35 parts of crataegin, 30 parts of gypenoside, and 40 parts of Panax notoginseng saponins, as well as 10 parts of lecithin and 60 parts of solid acetic acid, and added into the concentrated product and mixed homogeneously; and
h) drying: the mixture obtained from step g) was dried under microwave to provide a dry final product.

### Embodiment 7

### Animal Experiment

Experimental method: 10 mice were randomly divided into two groups of A and B with 5 for each. Group A was fed with a conventional feed added with the dry final product of the composition prepared in embodiment 5 of this invention. Group B as a comparative group was fed with a conventional feed.

The mice from each of groups A and B were fed three times per day. And they were observed for 30 days.

Experimental results: during the 30-day feeding, the mice from group A appeared more active with a relatively high sensitivity, and the mice from group B appeared less active.

It was found from the lipid analysis post 30 days that: HDL in the blood of the mice from group A was greater than that from group B by 32.53% on average, triglyceride in the blood of those from group A was less than that from group B by 27.16% on average, and the blood viscosity of those from group A was less than that from group B by 25%. Moreover, it was found from dissection that the elasticity of blood vessels was normal for the mice from group A, and for those from group B, the degree of vascular sclerosis was increased by above 15% and the thickness of blood vessel walls was increased by 13.2%. No damage to the liver or kidney was found in all of the 5 mice from group A after dissection.

### Embodiment 8

### Clinical Trials

Experimental method: 5 patients with hypertension, 5 patients with stroke and paralysis, and 5 patients with angina. Each of the patients was administered with the dry final product powder of the composition prepared in embodiment 4 of this invention, and took the medicine three times a day by 3 g for each with warm water for 8 courses of treatment in total, in which 30 days was deemed as one course of treatment.

Experimental Results:

Patients with Hypertension

5 patients with hypertension had a diastolic pressure of above 180 and a systolic pressure of above 110, with the chief complaints of dizziness, tinnitus, chest distress, short of breath, and insomnia, and with partial blockage appeared in the skull Doppler.

After 8 courses of treatment using the composition of this invention, the diastolic pressures of 3 patients therein were reduced to below 140, the systolic pressures thereof were around 100, the chief complaints disappeared, no vascular abnormality was shown in the skull Doppler, and the electrocardiogram showed that the blood supply for the heart and brain was normal; the diastolic pressure of another one patient was 150, the systolic pressure was 100, the chief complaints essentially disappeared, no vascular abnormality was shown in the skull Doppler, and the electrocardiogram showed that the blood supply for the heart and brain was normal; and it was ineffective for the remained one.

Patients with Stroke

5 patients had the symptoms of dottiness, eye-mouth deviation, and paralysis in bed, with one of them lying in bed for 1 year.

After 8 courses of treatment using the composition of this invention, 4 patients of them could get out of bed by themselves, walk slowly with a stick, and do some simple housekeeping independently; and 1 patient therein began to be sober-minded, could speak basically, and could raise both arms and legs by himself/herself.

Patients with Angina

5 patients had the symptoms of palpitation, short of breath, choking, irregular attacks of chest pain, panting on walking, and weakness of legs, with a severe shortage of blood supply appeared in the electrocardiogram.

One of the 5 patients was 75 years old, and thus was administered three times a day with 1.5 g for each for 4 courses of treatment, and the remained patients were normally administered for 8 courses of treatment. Among four (including the one of 75 years old above) of these patients, the symptoms substantially disappeared, and the electrocardiograms showed that the blood supply was normal; and in one of the five patients, the symptom of choking disappeared, the attack duration and frequency of chest pain were reduced, and the electrocardiogram showed that the blood supply was basically normal.

What is claimed is:

1. A plant extract composition functioning as a scavenger of blood vessels, mainly composed of plant raw materials and an extract, wherein the plant raw materials comprise the following components according to parts by weight: 10-30 parts of Folium Isatidis, 15-35 parts of Semen Cassiae, 10-20 parts of Flos Inulae, 15-30 parts of Flos Carthami, 10-30 parts of Flos Dendranthemae, and 20-50 parts of Folium Apocyni Veneti; and the extract comprises the following components according to parts by weight: 20-40 parts of Ginkgo flavone, 20-35 parts of emodin, 30-50 parts of crataegin, 15-55 parts of gypenoside, and 20-60 parts of Panax notoginseng saponins.

2. The plant extract composition of claim 1, wherein the plant raw materials comprise the following components according to parts by weight: 15-25 parts of Folium Isatidis, 20-30 parts of Semen Cassiae, 12-18 parts of Flos Inulae, 20-30 parts of Flos Carthami, 15-25 parts of Flos Dendranthemae, and 30-40 parts of Folium Apocyni Veneti; and the extract comprises the following components according to parts by weight: 25-35 parts of Ginkgo flavone, 25-35 parts of emodin, 30-40 parts of crataegin, 20-40 parts of gypenoside, and 30-50 parts of Panax notoginseng saponins.

3. The plant extract composition of claim 1, further comprising the following raw materials according to parts by weight: 10-30 parts of lecithin, 10-30 parts of black Auricularia, and 20-80 parts of solid acetic acid.

4. The plant extract composition of claim 2, further comprising the following raw materials according to parts by weight: 10-30 parts of lecithin, 10-30 parts of black Auricularia, and 20-80 parts of solid acetic acid.

5. The plant extract composition of any one of claims 1-4, wherein the formulation of the plant extract composition is capsule, tablet, powder, or granule.

6. A method for preparing the plant extract composition of any one of claims 1-4, comprising the following steps:
a) pulverizing: weighing the plant raw materials according to parts by weight respectively and pulverizing them;
b) soaking: the pulverized plant raw materials from step a) are placed into a sealed container, and then water is added thereto in a solid/liquid ratio of 1:4 by weight, and the resultant is soaked for 2-5 hours;
c) ultrasonic extracting: the plant raw materials and soak solution obtained from step b) are extracted in an ultrasonic tank, where the temperature was raised from a room temperature to no greater than 40° C., and the extraction time was 50-60 minutes;
d) filtrating and separating: the mixed solution obtained from step c) is filtered to obtain a filtrate and a residue, the residue is subjected to centrifugal filtration to obtain a secondary filtrate, and both the filtrates are combined into a final filtrate;
e) adjusting pH: the final filtrate is adjusted to having a pH of 7.5-7.7;
f) concentrating: the final filtrate is concentrated to a concentrated product having a content of 75-80%;
g) adding a plant extract: the plant extract is weighted according to parts by weight, added into the concentrated product and mixed homogeneously; and
h) drying: the mixture obtained from step g) is dried to provide a dry final product.

7. The method of claim 6, wherein in the step c), the stirring rate in the ultrasonic tank is 1500 rpm, the ultrasonic power is 1500 W, and the ultrasonic interval is 1.5 minutes.

8. The method of claim 6 or 7, wherein in the step h), vacuum drying or microwave drying is used.
